(19) United States
(12) Patent Application Publication
(10) Pub. No.: US 2026/0240738 A1
(43) Pub. Date: Aug. 20, 2026

(54) ORAL CARE PRODUCT COMPOSITIONS AND METHODS OF USE

(71) Applicant: University of Connecticut, Farmington, CT (US)

(72) Inventor: Marcus A. Costa, Farmington, CT (US)

(21) Appl. No.: 19/541,480

(22) Filed: Feb. 17, 2026

Related U.S. Application Data
(60) Provisional application No. 63/759,634, filed on Feb. 18, 2025.

(51) Int. Cl. A61K 8/46; A61K 8/21; A61K 8/24; A61Q 11/00

(52) U.S. Cl. CPC A61K 8/463; A61K 8/21; A61K 8/24; A61Q 11/00

(57) ABSTRACT

Provided herein are compositions and methods for promoting oral (e.g., dental) hygiene and/or oral microbiome balance, for example, by reducing plaque, whitening teeth, preventing tooth demineralization, and providing tooth remineralization. Specifically, provided herein are compositions (e.g., chewing gum or oral care) products and methods of using such products for oral care.

### RELATED APPLICATIONS

This application claims priority to provisional application 63/759,634, filed Feb. 18, 2025, which is herein incorporated by reference in its entirety.

### FIELD OF THE DISCLOSURE

Provided herein are compositions and methods for promoting oral (e.g., dental) hygiene. Specifically, provided herein are compositions (e.g., chewing gum or oral care compositions) and methods of using such products for oral care.

### BACKGROUND

The current solutions for maintaining oral hygiene throughout the day primarily involve brushing teeth or using mouthwash. Brushing, while effective, is often time-consuming and impractical in environments like schools and workplaces. Mouthwash is another option, but many are formulated to indiscriminately kill both harmful and beneficial bacteria in the mouth, potentially disrupting the oral microbiome.

Therefore, there is an urgent unmet need for new oral care alternatives that focus on supporting a balanced bacterial landscape and promoting oral hygiene.

### SUMMARY

The following brief summary is not intended to include all features and aspects of the present disclosure, nor does it imply that any claim must include all features and aspects discussed in this summary.

Provided herein are compositions and methods for promoting oral (e.g., dental) hygiene, for example, by reducing plaque, whitening teeth, preventing tooth demineralization, and promoting tooth remineralization. Specifically, provided herein are compositions (e.g., chewing gum or oral care) products and methods of using such products for oral care.

The compositions and methods of the present disclosure improve oral care by providing a composition in a convenient administration form that improves oral care, prevents dental caries, and improves the overall health of subjects.

In an aspect, disclosed is a chewing gum or oral care formulation comprising a basic amino acid (e.g., L-arginine), an alkali-generating or buffering system (e.g., a bicarbonate salt such as sodium bicarbonate), and hydroxyapatite, as shown and described herein.

In an aspect, disclosed is a method for improving oral health or hygiene as shown and described herein.

For example, in some embodiments, provided herein is a composition comprising L-arginine, a bicarbonate salts (e.g., sodium bicarbonate), and hydroxyapatite.

The present disclosure is not limited to particular bicarbonate salts. Examples include but are not limited to, as potassium bicarbonate, calcium bicarbonate, magnesium bicarbonate, ammonium bicarbonate, lithium bicarbonate, or any combinations thereof.

The present disclosure is not limited to particular compositions. In certain aspects, the composition is a chewing gum or an oral care product.

In some aspects, the composition comprises from about 0.01% to about 15% by weight of L-arginine, from about 0.1% to about 50% by weight of sodium bicarbonate, and from about 0.01% to about 30% by weight of hydroxyapatite (e.g., from about 0.5% to about 10% by weight of L-arginine, from about 0.5% to about 35% by weight of sodium bicarbonate, and from about 0.5% to about 20% by weight of hydroxyapatite or from about 0.01% to about 5% by weight of L-arginine, from about 0.1% to about 25% by weight of sodium bicarbonate, and from about 0.01% to about 15% by weight of hydroxyapatite). In some embodiments, the L-arginine and sodium bicarbonate are present in the composition at a weight ratio of about 1:4.

In certain embodiments, the composition comprises from about 10% to about 95% by weight gum base. The gum base may include any suitable components. Examples include but are not limited to elastomers (e.g., chicle, jelutong, gutta-percha, guayale, crown gum, butadiene-styrene copolymers, isobutylene-isoprene copolymers, polyethylene, polyisobutylene, polyvinyl acetate, or any combinations thereof), solvents, plasticizers, fillers, flavoring agents, coloring agents, and sweetening agents.

In some embodiments, the elastomers are present in the composition at a concentration of from about 14% to about 50% by weight (e.g., from about 20% to about 30% by weight).

In some aspects, the composition further comprises one or more elastomer solvents (e.g., methyl, glycerol, or pentaerythritol esters of rosins or modified (e.g., hydrogenated, dimerized, polymerized rosins, or any combinations thereof). In some embodiments, the elastomer solvent is present at a concentration of about 10% to about 75% by weight (e.g., about 15% to about 50% by weight).

Certain compositions further comprise a terpene resin.

In still further embodiments, the composition further comprises one or more additional components selected from lanolin, lecithin, glycerol monostearate, stearic acid, glycerol triacetate, triacetin, glycerin, natural waxes, petroleum waxes, microcrystalline waxes, lard, tallow, soybean oil, cottonseed oil, hydrogenated vegetable oil, partially hydrogenated vegetable oil and cocoa butter, wherein the additional components are present in the composition and a concentration of up to about 30% by weight.

In other embodiments, the composition comprises from about 10% to about 95% by weight of confectionery base.

In further aspects, the composition comprises a sweetener (e.g., sucrose, dextrose, maltose, fructose, aspartame, acesulfame salts, alitame saccharin, sorbitol, mannitol, xylitol, maltitol, isomalt, erythritol, hydrogenated starch hydrolysates, coconut oil, or combinations thereof).

Some compositions further comprise a coloring agent.

Certain compositions further comprise a filler selected from dicalcium phosphate, magnesium carbonate, aluminum hydroxide, alumina, aluminum silicates, talc, calcium carbonate, cellulose, or combinations thereof, wherein the filler is present in composition at a concentration of from about 5% to about 35% by weight.

In some cases, the composition further comprises one or more of a chemical whitening agent, an opaque whitening agent, an anticalculus agent, a surfactant system that comprises sodium lauryl sulfate and an N-acyl taurate (e.g., sodium methyl cocoyl taurate (SMCT)), a stannous ion agent; a fluoride compound, sodium fluoride, chlorhexidine, alexidine; hexetidine, sanguinarine, benzalkonium chloride, salicylanilide, domiphen bromide, cetylpyridinium chloride (CPC), tetradecyl pyridinium chloride (TPC), N-tetradecyl-4-ethylpyridinium chloride (TDEPC), octenidine, delmopinol, octapinol, nisin, zinc ion, copper ion, essential oils, furanones, bacteriocins, ethyllauroyl arginate, extracts of magnolia, a metal ion source, arginine bicarbonate, honokiol, magnolol, ursolic acid, tirsic acid, morin, extract of sea buckthorn, a peroxide, an enzyme, a camellia extract, a flavonoid, a flavin, halogenated diphenyl ether, creatine, propolis, or any combinations thereof.

Aspects of the disclosure provide a kit or system comprising a composition described herein.

Also provided is a method of improving oral health or hygiene, comprising: providing a composition described herein to a subject in need thereof (e.g., to an oral surface of the subject). In some embodiments, the providing reduces dental sensitivity in the subject, reduces the incidence of dental caries in the subject, reduces dental plaque in the subject, whitens teeth in the subject, prevents tooth demineralization in the subject, promotes tooth remineralization in the subject, improves overall health in the subject, or any combination thereof. In some cases, the subject is a human. In some embodiments, the subject is an orthodontic patient, a child, or a subject with cognitive motor impairments. In some embodiments, the subject is a companion animal or livestock.

Further provided is the use of a composition described herein to improve oral health or hygiene.

In certain aspects, the L-arginine is present as a salt selected from arginine bicarbonate, arginine carbonate, arginine citrate, arginine acetate, arginine lactate, arginine phosphate, arginine hydrochloride, or combinations thereof. In some embodiments, the bicarbonate salt further comprises a carbonate salt and/or a phosphate salt. In some embodiments, the hydroxyapatite is nano-hydroxyapatite having an average particle size of about 20 nm to about 900 nm. In certain aspects, the hydroxyapatite comprises fluorohydroxyapatite and/or fluorapatite. In some embodiments, one or more of (i) the L-arginine, (ii) the bicarbonate salt, and (iii) the hydroxyapatite is encapsulated or coated to provide staged release in the oral cavity.

In some embodiments, the composition (e.g., chewing gum) comprises a coating and wherein at least a portion of one or more of the L-arginine, bicarbonate salt, and hydroxyapatite is present in the coating.

In some embodiments, the composition further comprises one or more calcium and/or phosphate ion donors selected from, for example, calcium glycerophosphate, dicalcium phosphate, calcium lactate, calcium citrate, sodium phosphate, or combinations thereof.

In some embodiments, the composition is formulated as a lozenge, mint, chewable tablet, oral dissolvable film, or oral powder.

In some embodiments, providing the composition comprises chewing or dissolving the composition for at least about a minute (e.g., 1, 5, 10, 15, or 20 minutes). In some embodiments, providing the composition is performed after a meal or snack to reduce post-prandial plaque acidification. In certain aspects, providing the composition raises plaque and/or oral pH above about 5 (e.g., 5.5) within about 5 (e.g., 5, 10, 15, or 20) minutes of administration.

In some embodiments, the subject is a companion animal (e.g., dog or cat) and the composition is provided as an edible chew or treat.

Also provided is a method of making a chewing gum composition, comprising mixing at least a portion of hydroxyapatite with chewing gum additives to form a premix and combining the premix with a gum base, wherein the bicarbonate salt is added in dry form.

Further provided is the use of a composition described herein for promoting a balanced oral microbiome by reducing acidogenic and/or aciduric bacteria while maintaining one or more commensal oral bacteria.

These and other aspects of the present disclosure are described in more detail below.

### BRIEF DESCRIPTION OF THE DRAWINGS

The accompanying drawings are included to provide a further understanding of the methods and compositions of the disclosure, and are incorporated in and constitute a part of this specification. The drawings illustrate one or more embodiment(s) of the disclosure, and together with the description serve to explain the principles and operation of the disclosure.

FIG. 1 shows Cq averages of S. mutans (pathogenic), S. anginosus (pathogenic), S. salivarius (commensal in newborns), and S. sanguinis (commensal in adults) after contact with compositions of embodiments of the present disclosure.

FIG. 2 shows genome copy data of S. mutans (pathogenic), S. anginosus (pathogenic), S. salivarius (commensal in newborns), and S. sanguinis (commensal in adults) after contact with compositions of embodiments of the present disclosure.

### DETAILED DESCRIPTION

Before the disclosed processes and devices are described, it is to be understood that the aspects described herein are not limited to specific embodiments, or examples, and as such can, of course, vary. It is also to be understood that the terminology used herein is for the purpose of describing particular aspects only and, unless specifically defined herein, is not intended to be limiting.

Provided herein are compositions and methods for promoting oral (e.g., dental) hygiene and general health, for example, by reducing plaque, whitening teeth, preventing tooth demineralization, and promoting tooth remineralization. Specifically, provided herein are compositions (e.g., chewing gum or oral care) products and methods of using such products for oral care.

Many individuals rely on conventional oral hygiene methods that often eliminate both harmful and beneficial microorganisms, leading to imbalances in the oral microbiome. This indiscriminate approach can be especially problematic for populations with unique challenges, such as orthodontic patients, children, or those with cognitive motor impairments, who may struggle to maintain consistent oral care or access specialized products. Additional products for oral care for companion animals and livestock are also needed. Traditional interventions like brushing and mouthwash depend on user compliance and may not always address underlying microbiome needs, while more advanced options, including probiotic formulations with live bacteria, can be expensive and require strict storage conditions to remain effective. Studies in microbial ecology underscore the importance of preserving beneficial bacteria to support oral health, highlighting growing need for cost-efficient, convenient, and microbiome-supporting solutions that can be used throughout the day.

The present disclosure addresses these needs by providing oral care products (e.g., chewing gum or other oral care product compositions for use in human or non-human animals), which reduce plaque, whiten teeth, prevent tooth demineralization, and provide tooth remineralization in the oral cavity. The disclosed formulations, in some embodiments, comprise as active ingredients, a combination of L-arginine, a bicarbonate salt such as sodium bicarbonate, and hydroxyapatite. The present disclosure overcomes the problem of combining the ingredients in an oral delivery system by use of a solid system such as a chewing gum or an oral care composition (e.g., confection). The terms chewing gum formulation/composition and oral care product formulation/composition are used interchangeably herein.

For example, in some embodiments, disclosed herein is a chewing gum composition/formulation that supports a balanced oral microbiome by combining L-arginine and sodium bicarbonate, which work together to neutralize plaque acids and buffer pH levels and provide prebiotics for oral health. In an embodiment, the formulation may also include hydroxyapatite for strengthening tooth enamel, and all components remain stable at room temperature in powder form. In some embodiments, the compositions find use in populations with oral hygiene challenges such as orthodontic patients, children, or individuals with inconsistent care routines, providing a natural, cost-effective alternative that targets harmful bacteria without disrupting overall oral flora. In some embodiments, the compositions reduce pathogenic bacteria while preserving commensals.

In further embodiments, the compositions find use in animal health and veterinary populations, including companion animals, livestock, and working animals that experience oral hygiene challenges due to limited access to routine dental care, behavioral constraints, age, or diet. In such applications, the compositions provide a natural, cost-effective approach for managing oral microbial load, selectively targeting pathogenic or malodor-associated microorganisms while preserving beneficial oral flora, thereby supporting overall oral and systemic health in non-human animals.

In some embodiments, the compositions are suitable for administration to animals, including mammals and non-mammalian species, for the prevention or management of oral conditions associated with pathogenic microbial growth, plaque formation, gingivitis, periodontal disease, or halitosis.

Some unique advantages of the compositions and methods disclosed herein include 1) targeting of oral microbiome balance: Unlike broad-spectrum mouthwashes, which kill both harmful and beneficial bacteria, the compositions and methods of the present disclosure support a healthier overall bacterial community through a prebiotic formulation; 2) cost-effectiveness over live probiotic products: Compared to formulations that use live bacteria, the compositions and methods of the present disclosure uses stabilized prebiotics (e.g., L-arginine, sodium bicarbonate), reducing manufacturing and cold-chain storage costs; 3) convenient for orthodontic and special needs populations: Whereas manual brushing or mouthwashes can be challenging for orthodontic patients or those with limited dexterity, the compositions and methods of the present disclosure can be used easily throughout the day for ongoing oral care support; 4) ingredient combination: Traditional baking soda gums do not incorporate L-arginine and hydroxyapatite. The unique blend of components of the compositions and methods of the present disclosure enhances neutralization and remineralization in a single product; 5) stable and shelf-ready formulation: Unlike live probiotic products requiring specific storage conditions, all ingredients in the compositions and methods of the present disclosure remain effective at room temperature simplifying distribution and retail placement; and 6) redefinition of the chewing gum industry: The available sugar-free gums focus on sweetening and masking odors; the compositions and methods of the present disclosure offer a functional oral care benefit that enhances market offerings.

The following terms are used to describe the present disclosure. In instances where a term is not specifically defined herein, that term is given an art-recognized meaning by those of ordinary skill applying that term in context to its use in describing the present disclosure.

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well-known and commonly used in the art. In case of conflict, the present disclosure, including definitions, will control. Exemplary methods and materials are described below, although methods and materials similar or equivalent to those described herein can be used in practice or testing of the embodiments and aspects described herein.

Compounds and materials are described using standard nomenclature. Unless defined otherwise, all technical and scientific terms used herein have the same meaning as is commonly understood by one of skill in the art to which this disclosure belongs. The following terms are used to describe the present disclosure. In instances where a term is not specifically defined herein, that term is given an art-recognized meaning by those of ordinary skill applying that term in context to its use in describing the present disclosure.

As used herein, “oral care product” refers to any product intended for delivery to the oral cavity to provide a cosmetic, hygienic, therapeutic, prophylactic, or diagnostic benefit. Non-limiting examples include, but are not limited to, chewing gum, confections (hard or soft), lozenges, mints, chewable tablets, oral dissolvable films or strips, oral powders, dentifrices, gels, mouthrinses, sprays, varnishes, and pet chews or chew toys that release active agents during chewing.

As used herein, “basic amino acid” includes arginine, lysine, histidine, and salts, derivatives, and mixtures thereof. In certain embodiments, the basic amino acid is L-arginine. As used herein, “arginine” includes L-arginine, D-arginine, racemic mixtures, and salts thereof (e.g., bicarbonate, carbonate, hydrochloride, acetate, citrate, lactate, malate, phosphate, sulfate), as well as arginine-containing complexes with calcium salts or phosphate salts.

As used herein, “hydroxyapatite” includes substituted apatites and calcium phosphate materials capable of depositing on, binding to, or otherwise interacting with enamel or dentin to support remineralization or reduce sensitivity. Non-limiting examples include hydroxyapatite, nano-hydroxyapatite, calcium-deficient hydroxyapatite, carbonate-substituted hydroxyapatite, fluorohydroxyapatite, fluorapatite, and mixtures thereof.

As used herein, “prebiotic” refers to a substrate that is selectively utilized by host microorganisms conferring a health benefit. In certain embodiments, arginine is utilized by arginolytic oral bacteria to generate alkali and counteract acidification of dental plaque.

The use of the terms “a” and “an” and “the” and similar referents (especially in the context of the following claims) are to be construed to cover both the singular and the plural, unless otherwise indicated herein or clearly contradicted by context. By way of example, “an element” means one element or more than one element.

As used herein, the term “substantially” means to a great or significant extent, but not completely.

It should also be understood that, in certain methods described herein that include more than one step or act, the order of the steps or acts of the method is not necessarily limited to the order in which the steps or acts of the method are recited unless the context indicates otherwise. Furthermore, the terms first, second, etc., as used herein are not meant to denote any particular ordering, but simply for convenience to denote a plurality of, for example, layers.

The terms “comprising”, “having”, “including”, and “containing” are to be construed as open-ended terms (i.e., meaning “including, but not limited to”) unless otherwise noted.

The terms “about” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. All ranges disclosed herein include both end points as discrete values as well as all integers and fractions specified within the range. For example, a range of 0.1-2.0 includes 0.1, 0.2, 0.3, 0.4 . . . 2.0. All methods described herein can be performed in a suitable order unless otherwise indicated herein or otherwise clearly contradicted by context. The use of any and all examples, or exemplary language (e.g., “such as”), is intended merely to better illustrate the disclosure and does not pose a limitation on the scope of the disclosure unless otherwise claimed. No language in the specification should be construed as indicating any non-claimed element as essential to the practice of the disclosure as used herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “one or more,” as used herein, means at least one, and thus includes individual components as well as mixtures/combinations of the listed components in any combination.

Other than in the operating examples, or where otherwise indicated, all numbers expressing quantities of ingredients and/or reaction conditions are to be understood as being modified in all instances by the term “about,” meaning within 10% of the indicated number (e.g., “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

As used herein, “optional” or “optionally” means that the subsequently described event or circumstance may or may not occur, and that the description includes instances where said event or circumstance occurs and instances where it does not.

The term “subject” or “patient” is used herein to refer to an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, and a whale), a bird (e.g., a duck or a goose), and a shark. In an embodiment, the subject or patient is a human subject or a human patient, such as a human being treated or assessed for a disease, disorder or condition, a human at risk for a disease, disorder or condition, a human having a disease, disorder or condition, and/or human being treated for a disease, disorder or condition as described herein. In one embodiment, the subject is about 1, 2, 3, 4, 5, 6, 7, 8, 9, or 10 years of age. In another embodiment, the subject is about 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100 years of age. Values and ranges intermediate to the above recited ranges are also intended to be part of this disclosure. In addition, ranges of values using a combination of any of the above-recited values as upper and/or lower limits are intended to be included. As used herein, a subject is “in need of treatment” if such subject would benefit biologically, medically, or in quality of life from such treatment. A subject in need of treatment does not necessarily present symptoms, particular in the case of preventative or prophylaxis treatments. The terms “subject” and “patient” are used interchangeably herein. The term “subject” also refers to an organism, tissue, cell, or collection of cells from a subject.

All methods described herein can be performed in a suitable order unless otherwise indicated herein or otherwise clearly contradicted by context. The use of any and all examples, or exemplary language (e.g., “such as”), is intended merely to better illustrate the disclosure and does not pose a limitation on the scope of the disclosure unless otherwise claimed. No language in the specification should be construed as indicating any non-claimed element as essential to the practice of the disclosure as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

All compounds are understood to include all possible isotopes of atoms occurring in the compounds. Isotopes include those atoms having the same atomic number but different mass numbers and encompass heavy isotopes and radioactive isotopes. By way of general example, and without limitation, isotopes of hydrogen include tritium and deuterium, and isotopes of carbon include ¹¹C, ¹³C, and ¹⁴C. Accordingly, the compounds disclosed herein may include heavy or radioactive isotopes in the structure of the compounds or as substituents attached thereto. Examples of useful heavy or radioactive isotopes include ¹⁸F, ¹⁵N, ¹⁸⁰, ⁷⁶Br, ¹²⁵I and ¹³¹I.

A significant change is any detectable change that is statistically significant in a standard parametric test of statistical significance such as Student's t-test or analysis of variance, where p<0.05.

### Formulations/Compositions

In an aspect, disclosed is a chewing gum or oral care product composition or formulation including a basic amino acid (e.g., L-arginine), an alkali-generating or buffering system (e.g., a bicarbonate salt such as sodium bicarbonate), and hydroxyapatite. In an embodiment, the chewing gum, confection, or other oral care composition comprises L-arginine, a bicarbonate salt (e.g., sodium bicarbonate, potassium bicarbonate, calcium bicarbonate, magnesium bicarbonate, ammonium bicarbonate), and hydroxyapatite. In an embodiment, the chewing gum, confection, or other oral care composition re-establishes oral microbiome homeostasis. In an embodiment, the bicarbonate salt is sodium bicarbonate. In an embodiment, the oral care product formulation is a confectionery. In an aspect, disclosed is a composition comprising L-arginine, a bicarbonate salt (e.g., sodium bicarbonate), and hydroxyapatite, wherein the composition is formulated for oral administration and is effective to reduce plaque and promote remineralization.

In some embodiments, the composition comprises a basic amino acid (e.g., arginine, lysine, or histidine), an alkali-generating or buffering system (e.g., bicarbonate and/or carbonate salts, phosphate salts, borate salts, citrate salts), and a calcium phosphate phase (e.g., hydroxyapatite) in any suitable combination. In certain embodiments, the basic amino acid is L-arginine, the buffering system comprises sodium bicarbonate, and the calcium phosphate phase comprises hydroxyapatite.

In some embodiments, the basic amino acid is provided as a salt (e.g., arginine bicarbonate, arginine carbonate, arginine phosphate, arginine citrate) or as a complex with a calcium salt (e.g., calcium carbonate) to provide enhanced substantivity to enamel and/or improved buffering.

In some embodiments, the hydroxyapatite has an average particle size of less than about 1 micron (e.g., about 20 nm to about 900 nm (e.g., about 20 nm, 50 nm, 100 nm, 200 nm, 400 nm, 500 nm, 700 nm, 800 nm, or 900 nm)) or from about 1 micron to about 20 microns, and may be surface-treated (e.g., with silica, surfactant, polymer, or lipid) to improve dispersibility in the product matrix.

In some embodiments, one or more of the active ingredients are coated or encapsulated (e.g., polymer-coated bicarbonate particles, lipid-encapsulated arginine, or coated hydroxyapatite) to improve stability during processing and storage and/or to provide staged release during chewing or dissolution. In certain embodiments, the composition includes a first portion of an active in a gum base and a second portion of the same or different active in a coating, center fill, or microcapsules.

In some embodiments, the composition further includes one or more calcium and/or phosphate ion donors (e.g., calcium glycerophosphate, dicalcium phosphate dihydrate, calcium lactate, calcium citrate, sodium phosphate) to support remineralization and/or to provide co-precipitation with hydroxyapatite on enamel.

In some embodiments, the composition is formulated to raise plaque pH above about 5 (e.g., 5.5) within about 1 to about 30 (e.g., 1, 5, 10, 15, 20, 25, or 30) minutes after administration and/or to maintain plaque pH above about 5.5 for at least about 10 minutes, at least about 20 minutes, or at least about 30 minutes following an acid challenge.

In an embodiment, the chewing gums or oral care product formulations may contain from about 0.01% to about 15% by weight of L-arginine, from about 0.1% to about 50% by weight of sodium bicarbonate, and from about 0.01% to about 30% by weight of hydroxyapatite. In an embodiment, the chewing gums or oral care product formulations may contain from about 0.5% to about 10% by weight of L-arginine, from about 0.5% to about 35% by weight of sodium bicarbonate, and from about 0.5% to about 20% by weight of hydroxyapatite.

In an embodiment, the chewing gums and confections may contain from about 0.01% to about 5% by weight of L-arginine. In an embodiment, the chewing gums or oral care product formulation may contain from about 0.1% to about 25% by weight of sodium bicarbonate. In an embodiment, the chewing gums or oral care product formulations may contain from about 0.01% to about 15% by weight of hydroxyapatite. In some embodiments, the L-arginine and sodium bicarbonate are present in a weight ratio of about 1:4.

In an embodiment, the chewing gum formulation includes from about 10% to about 95% by weight gum base. In an embodiment, the confectionery product formulation includes from about 10% to about 95% by weight confectionery base. In an embodiment, the chewing gum formulation can be sugar-free. In an embodiment, the chewing gum formulation can contain sugar. In an embodiment, the chewing gum formulation comprises one or more natural or synthetic elastomers which are supplemented by conventional chewing gum ingredients. In an embodiment, the chewing gum formulation includes one or more solvents, plasticizers, fillers, flavoring agents, coloring agents, sweetening agents, or any combinations thereof. In an embodiment, the elastomers which are suitable for use herein include substances of vegetable origin such as chicle, jelutong, gutta percha, guayule and crown gum. In an embodiment, the elastomers include synthetic elastomers such as butadiene-styrene copolymers, isobutylene-isoprene copolymers, polyethylene, polyisobutylene, polyvinyl acetate, or any combinations thereof. In an embodiment, the elastomer generally comprises from about 14% to about 50% by weight, preferably from about 20% to about 30% by weight, of the chewing gum composition. In an embodiment, polyvinyl acetates may also be used with the elastomers to provide stretch or elasticity to the gum.

In an embodiment, the chewing gum composition can contain elastomer solvents to aid in softening the polymer component. Some nonlimiting examples of elastomer solvents include methyl, glycerol or pentaerythritol esters of rosins or modified rosins, such as hydrogenated, dimerized or polymerized rosins, or any combinations thereof. In an embodiment, the chewing gum composition includes terpene resins, including polyterpene and mixtures thereof. In an embodiment, the solvent can be employed in an amount ranging from about 10% to 75% and preferably about 15% to about 50% by weight of the chewing gum composition.

In an embodiment, the chewing gum composition includes plasticizers or emulsifiers such as lanolin, lecithin, glycerol monostearate, stearic acid, glycerol triacetate, triacetin, glycerin and the like. These ingredients can be incorporated into the chewing gum composition to obtain a variety of textures and consistency properties. In an embodiment, the chewing gum composition also may include waxes such as natural waxes, petroleum waxes and microcrystalline waxes and fats and oils including animal fats such as lard and tallow, vegetable oils such as soybean and cottonseed oil, hydrogenated and partially hydrogenated vegetable oil and cocoa butter. These ingredients are generally employed in amounts of up to about 30% by weight, preferably about 1% to about 25% by weight and more preferably from about 1% to about 5% by weight of the final chewing gum composition.

In certain embodiments, the composition can additionally include coloring agents such as titanium dioxide, in amounts up to about 2% and fillers such as dicalcium phosphate, magnesium carbonate, aluminum hydroxide, alumina, aluminum silicates, talc, calcium carbonate, cellulose, and combinations thereof in amounts of from about 5% to about 35% by weight of the final composition.

In an embodiment, the composition may also contain bulk sweeteners including sugars such as sucrose, dextrose, maltose, fructose, and the like, or sugar alcohols such as sorbitol, mannitol, xylitol, maltitol, isomalt, erythritol, and hydrogenated starch hydrolysates, or coconut oil, or any combinations thereof. Bulk sweeteners may be present in amounts up to about 90% by weight of the final composition. In certain embodiments, sweeteners such as aspartame, acesulfame salts, alitame saccharin, or any combination thereof may also be present. These sweeteners may be present in amounts of up to about 1% by weight of the final gum composition.

In an embodiment, the composition may contain flavoring agents in amounts up to about 5%. Generally, any suitable food additive, such as those described in “Chemicals Used In Food Processing”, publication 1274, pages 63-258, by the National Academy of Sciences, may be used.

In an embodiment, the composition further comprises an agent including a chemical whitening agent, an opaque whitening agent, an anticalculus agent, or any combination thereof. In an embodiment, the chewing gum composition further comprises a surfactant system that comprises sodium lauryl sulfate and an N-acyl taurate (e.g., sodium methyl cocoyl taurate (SMCT) or TAURANOL surfactant). In an embodiment, a surfactant system consists essentially of sodium lauryl sulfate and the N-acyl taurate in a ratio of about 1:5 to 1:3.

In an embodiment, the composition further includes an agent including a stannous ion agent; a fluoride compound; sodium fluoride; chlorhexidine; alexidine; hexetidine; sanguinarine; benzalkonium chloride; salicylanilide; domiphen bromide; cetylpyridinium chloride (CPC); tetradecyl pyridinium chloride (TPC); N-tetradecyl-4-ethylpyridinium chloride (TDEPC); octenidine; delmopinol; octapinol; nisin; zinc ion agent; copper ion agent; essential oils; furanones; bacteriocins, ethyllauroyl arginate, extracts of magnolia, a metal ion source, arginine bicarbonate, honokiol, magnolol, ursolic acid, tirsic acid, morin, extract of sea buckthorn, a peroxide, an enzyme, a camellia extract, a flavonoid, a flavin, halogenated diphenyl ether, creatine, propolis, or any combinations thereof.

In an embodiment, the composition may further include any suitable therapeutic, cosmetic, and/or aesthetic materials as may be desired. Some nonlimiting examples include silica and/or non-silica desensitizing agents (for example, a nitrate salt, an arginine ester, a carbonate salt, potassium nitrate, an arginine-bicarbonate-phytate complex, potassium citrate, and arginine, among others), a chemical whitening agent (such as a peroxide releasing compound), an opaque whitening agent (such as hydroxyapatite), an anticalculus agent, or any combinations thereof. In some embodiments, the chewing gum composition may include triclosan; stannous ion agents; chlorhexidine; alexidine; hexetidine; sanguinarine; benzalkonium chloride; salicylanilide; domiphen bromide; cetylpyridinium chloride (CPC); tetradecyl pyridinium chloride (TPC); N-tetradecyl-4-ethylpyridinium chloride (TDEPC); octenidine; delmopinol; octapinol; nisin; zinc ion agents; copper ion agents; essential oils; furanones; bacteriocins, ethyl lauroyl arginate, extracts of magnolia, a metal ion source, arginine bicarbonate, honokiol, magnolol, ursolic acid, ursic acid, morin, extract of sea buckthorn, an enzyme, a camellia extract, a flavonoid, a flavin, halogenated diphenyl ether, creatine, propolis, or any combinations thereof.

In an embodiment, the composition may contain any suitable excipients or carriers. Some non-limiting excipients or carriers can include, for example, humectants, colorants, flavorings, glycerin, sorbitol, xylitol, and/or propylene glycol, water or other solvents, gum bases, thickening agents, surfactants, carrageenan (rich moss), xanthan gum and sodium carboxymethyl cellulose, starch, polyvinyl pyrrolidone, hydroxyethyl propyl cellulose, hydroxy butyl methyl cellulose, hydroxypropyl methyl cellulose, hydroxyl ethyl cellulose and amorphous silicas, or any combinations thereof.

In an embodiment, any suitable surfactant may be included, if desired. Some nonlimiting examples of suitable surfactants include water-soluble salts of higher fatty acid monoglyceride monosulfates, such as the sodium salt of monosulfated monoglyceride of hydrogenated coconut oil fatty acids; higher alkyl sulfates such as sodium lauryl sulfate; alkyl aryl sulfonates such as sodium dodecyl benzene sulfonate; higher alkyl sulfoacetates, such as sodium lauryl sulfoacetate; higher fatty acid esters of 1,2-dihydroxypropane sulfonate; and the substantially saturated higher aliphatic acyl amides of lower aliphatic amino carboxylic compounds, such as those having 12-16 carbons in the fatty acid, alkyl or acyl radicals; or any combinations thereof. Some nonlimiting examples amides include N-lauryl sarcosine, and the sodium, potassium and ethanolamine salts of N-lauryl, N-myristoyl, N-palmitoyl sarcosine, or any combinations thereof. Others examples include, for example, nonanionic polyoxyethylene surfactants, such as Polyoxamer 407, Steareth 30, Polysorbatc 20, and castor oil; and amphoteric surfactants, such as cocamidopropyl betaine (tegobaine), and cocamidopropyl betaine lauryl glucoside; condensation products of ethylene oxide with various hydrogen containing compounds that are reactive therewith and have long hydrocarbon chains (e.g., aliphatic chains of from 12 to 20 carbon atoms), which condensation products (ethoxamers) contain hydrophilic polyoxyethylene moieties, such as condensation products of poly (ethylene oxide) with fatty acids, fatty alcohols, fatty amides and other fatty moieties, with propylene oxide and polypropylene oxides, or any combinations thereof.

While not limited to particular formulations, in some exemplary embodiments, provided herein is a composition comprising L-arginine, sodium bicarbonate, and hydroxyapatite.

The present disclosure is not limited to particular compositions. In certain aspects, the composition is a chewing gum or an oral care product.

In further embodiments, the composition is formulated as an animal care product. Examples include but are not limited to, a chew toy infused or coated with a composition of embodiments of the present disclosure (L-arginine, sodium bicarbonate, hydroxyapatite) to promote oral health in animals (e.g., companion animals such as dogs or cats) while they chew. In an embodiment, the composition is formulated as a polymer matrix or porous structure that slowly releases active ingredients during chewing.

In an embodiment, the composition is a soft or hard edible chews for a companion animal or livestock that incorporate the composition into the bulk material (optionally flavored to encourage compliance and provide mechanical cleaning plus chemical benefits).

In certain embodiments, the composition is provided in a powdered or liquid form of the and is added to drinking water for animals (e.g., livestock) to reduce oral bacterial load and support dental health.

In other embodiments, the composition is provided as a gel formulation applied to teeth and gums of pets or livestock during routine care (e.g., by veterinarians or owners).

Also provided is a sprayable solution containing the composition for easy application to the oral cavity of animals that resist brushing such as companion animals.

In further embodiments, the composition is provided as a tablet or biscuit for small animals (e.g., rabbits, rodents) that incorporates the composition for oral health maintenance.

In one exemplary embodiment, the present disclosure provides a dog oral-health product (e.g., chew) comprising hydroxyapatite, a bicarbonate salt (e.g., sodium bicarbonate), L-arginine, and coconut-derived fatty acids. Each ingredient supports a different part of oral hygiene in dogs.

For example, bicarbonate can raise the pH in the mouth quickly by neutralizing acids. L-arginine can help beneficial oral bacteria create ammonia, which keeps the pH higher for a longer period of time (See e.g., Lindinger, Scientifica Volume 2016, Article ID 2183623).

Hydroxyapatite can attach to the tooth surface, strengthen enamel, and make it harder for plaque to adhere (See e.g., Tanongpitchayes et al., Vet Sci. 2021 Dec. 26; 9 (1):7. doi: 10.3390/vetsci9010007). The fatty acids in coconut oil, especially lauric acid, can interfere with the growth of harmful bacteria. Because each ingredient works through a different mechanism, the overall outcome may include reduced plaque, a healthier oral microbiome, stronger enamel surfaces, less halitosis, and a more stable oral pH.

Formulations for use in dogs typically remove xylitol, as is it not safe for dogs.

These ingredients may be added to a dog dental chew using standard treat-manufacturing processes. For example, in some embodiments, the hydroxyapatite is mixed directly into the chew as a fine powder. The sodium bicarbonate is included in the main mixture in its dry form. Bicarbonate should be kept dry during the mixing and shaping process so that it does not react prematurely. L-arginine is added in the same way. The coconut-derived fatty acids are added as part of the fat component of the chew. They can either be blended directly into the mixture or included as small encapsulated droplets to help them stay stable and maintain their antimicrobial effect.

The chew is not limited to particular forms. Examples include but are not limited to, a hard chew, a soft chew, a dental stick, or a compressed chew. All of these forms allow the ingredients to be released while the dog chews, creating friction that helps remove plaque at the same time, as seen in current market options.

Additional forms include but are not limited to a soft chew, a treat stick, a biscuit-type treat, a powder that can be sprinkled on food, or a coating on regular kibble.

The chew would be designed for daily use in a similar manner to existing dental sticks, but with an improved set of functional ingredients.

In some specific exemplary aspects, the composition comprises from about 0.01% to 15% by weight of L-arginine, from about 0.1% to about 50% by weight of sodium bicarbonate, and from about 0.01% to about 30% by weight of hydroxyapatite (e.g., from about 0.5% to about 10% by weight of L-arginine, from about 0.5% to about 35% by weight of sodium bicarbonate, and from about 0.5% to about 20% by weight of hydroxyapatite or from about 0.01% to about 5% by weight of L-arginine, from about 0.1% to about 25% by weight of sodium bicarbonate, and from about 0.01% to about 15% by weight of hydroxyapatite). In some embodiments, the L-arginine and sodium bicarbonate are present in the composition at a weight ratio of about 1:4.

In certain embodiments, the composition comprises from about 10% to about 95% by weight gum base. The gum base may include any suitable components. Examples include but are not limited to elastomers (e.g., chicle, jelutong, gutta percha, guayale, crown gum, butadiene-styrene copolymers, isobutylene-isoprene copolymers, polyethylene, polyisobutylene, polyvinyl acetate, or any combinations thereof), solvents, plasticizers, fillers, flavoring agents, coloring agents, and sweetening agents.

In some embodiments, the elastomers are present in the composition at a concentration of from about 14% to 50% by weight (e.g., from about 20% to about 30% by weight).

In some aspects, the composition further comprises one or more elastomer solvents (e.g., methyl, glycerol, or pentaerythritol esters of rosins or modified (e.g., hydrogenated, dimerized, polymerized rosins, or any combinations thereof). In some embodiments, the elastomer solvent is present at a concentration of about 10% to about 75% by weight (e.g., about 15% to about 50% by weight).

Certain compositions further comprise a terpene resin.

In still further embodiments, the composition further comprises one or more additional components selected from lanolin, lecithin, glycerol monostearate, stearic acid, glycerol triacetate, triacetin, glycerin, natural waxes, petroleum waxes, microcrystalline waxes, lard, tallow, soybean oil, cottonseed oil, hydrogenated vegetable oil, partially hydrogenated vegetable oil and cocoa butter, wherein the additional components are present in the composition and a concentration of up to about 30% by weight.

In other embodiments, the composition comprises from about 10% to about 95% by weight confectionery base.

In further aspects, the composition comprises a sweetener (e.g., sucrose, dextrose, maltose, fructose, aspartame, acesulfame salts, alitame saccharin, sorbitol, mannitol, xylitol, maltitol, isomalt, erythritol, hydrogenated starch hydrolysates, coconut oil, or combinations thereof).

Some compositions further comprise a coloring agent.

Certain compositions further comprise a filler selected from dicalcium phosphate, magnesium carbonate, aluminum hydroxide, alumina, aluminum silicates, talc, calcium carbonate, cellulose, or combinations thereof, wherein the filler is present in composition at a concentration of from about 5% to about 35% by weight.

In some cases, the composition further comprises one or more of a chemical whitening agent, an opaque whitening agent, an anticalculus agent, a surfactant system that comprises sodium lauryl sulfate and an N-acyl taurate, a stannous ion agent; a fluoride compound, sodium fluoride, chlorhexidine, alexidine; hexetidine, sanguinarine, benzalkonium chloride, salicylanilide, domiphen bromide, cetylpyridinium chloride (CPC), tetradecyl pyridinium chloride (TPC), N-tetradecyl-4-ethylpyridinium chloride (TDEPC), octenidine, delmopinol, octapinol, nisin, zinc ion, copper ion, essential oils, furanones, bacteriocins, ethyllauroyl arginate, extracts of magnolia, a metal ion source, arginine bicarbonate, honokiol, magnolol, ursolic acid, tirsic acid, morin, extract of sea buckthorn, a peroxide, an enzyme, a camellia extract, a flavonoid, a flavin, halogenated diphenyl ether, creatine, propolis, or any combinations thereof.

The chewing gum is generally manufactured by methods known in the art by sequentially adding the various chewing gum components to any commercial mixer or extruder in a batch or continuous process. After the ingredients have been thoroughly mixed the mass is discharged and formed.

The preparation of confectionery formulations is historically well known. In general, a hard boiled candy confection has a base composed of a mixture of sugar and other carbohydrate bulking agents kept in an amorphous or glassy condition having from about 0.5% to about 5% moisture. A general discussion of the composition and preparation of hard confections may be found in E. B. Jackson, Ed. “Sugar Confectionery Manufacture”. 2nd edition, Blackic Academic & Professional Press, Glasgow UK, (1990), at pages 129-169.

Such confectionery may be routinely prepared by conventional methods such as those involving fire cookers, vacuum cookers, and scraped-surface cookers, also referred to as high-speed atmospheric cookers. Once the candy mass has been properly tempered, it may be cut into workable portions or formed into desired shapes. A variety of forming techniques may be utilized depending upon the shape and size of the final product desired.

Soft candy confectionery includes fondants, caramels toffees, fudge, marshmallows and nougats and the like and may also include jams and jellies. The preparation of soft confections, such as nougat, involves conventional methods, such as the combination of two primary components, namely (1) a high boiling syrup such as a corn syrup, or the like, and (2) a relatively light textured frappe, generally prepared from egg albumin, gelatin, vegetable proteins, such as soy derived compounds, milk derived compounds such as milk proteins, and mixtures thereof. A general discussion of the composition and preparation of such confections may be found in E. B. Jackson. Ed. “Sugar Confectionery Manufacture”. 2nd edition. Blackie Academic & Professional Press. Glasgow UK. (1990), at pages 170-235.

Compressed tablet confections contain particular materials and are formed into structures under pressure. These confections generally contain sugars or sugar substitutes in amounts up to about 95%, by weight of the composition, and typical tablet excipients such as binders and lubricants as well as the enhanced flavoring agent, flavoring agents, colorants and so forth.

Aspects of the disclosure provide a kit or system comprising a composition described herein. In some embodiments, the kit or system comprising packing comprising one or more unit dosages of the described compositions (e.g., pieces of chewing gum, animal chews, or confections). In some embodiments, the kit or system comprises a measuring device (e.g., for delivering an appropriate amount of a liquid formulation such as a mouthwash).

### Methods

In an aspect, provided is a method for improving oral health or hygiene, the method including providing a chewing gum or an oral care product formulation (for example, confectionary), wherein the chewing gum or a confectionery formulation includes a basic amino acid (e.g., L-arginine), an alkali-generating or buffering system (e.g., a bicarbonate salt such as sodium bicarbonate), and hydroxyapatite. In an embodiment, the method includes providing a chewing gum or an oral care product formulation, wherein the chewing gum or a confectionery formulation includes L-arginine, a bicarbonate salt, and hydroxyapatite. In an embodiment, the bicarbonate salt is sodium bicarbonate.

In an embodiment, the method includes placing the chewing gum or a confectionery in the oral cavity, wherein the chewing gum or a confectionery includes L-arginine, a bicarbonate salt, and hydroxyapatite. In an embodiment, the method re-establishes oral microbiome homeostasis. In an embodiment, the method includes providing and consuming the chewing gum or confection after having food such as lunch or dinner. The present disclosure is not limited to particular compositions. In certain aspects, the composition is formulated as a chewing gum or another oral care product suitable for administration to the oral cavity.

In some embodiments, the composition is administered after eating and/or drinking (e.g., within about 0 to about 30 (e.g., 0 to 10) minutes after a meal or snack) to counteract post-prandial acidification of plaque.

In some embodiments, the composition is administered one or more times per day (e.g., 1, 2, 3, 4, or more times per day) for a duration of at least about 1 week, 2 weeks, 4 weeks, 8 weeks, 12 weeks, or longer.

In some embodiments, a unit dose comprises from about 10 mg to about 500 mg of L-arginine (or an equivalent amount of an arginine salt), from about 25 mg to about 2 g of a bicarbonate salt, and from about 10 mg to about 1 g of hydroxyapatite, depending on product format (e.g., gum piece, lozenge, chew, or treat). In some embodiments, the composition is formulated for sustained contact with teeth and/or plaque (e.g., chewing for at least about 5 minutes, at least about 10 minutes, or at least about 20 minutes).

In some embodiments, the disclosure provides compositions and methods for improving oral health in a companion animal and/or livestock. Examples of companion animals include but are not limited to, dogs, cats, rabbits, ferrets, guinea pigs, birds, hedgehogs, etc. Examples of livestock include but are not limited to, horses, cattle, sheep, pigs, poultry (e.g., chickens, turkeys), llamas, alpacas, bison, and farmed exotic/alternative animals (e.g., elk, ostriches), etc.

In an aspect, the disclosure provides compositions and methods for reducing dental sensitivity. In an aspect, a method of reducing dental sensitivity includes providing a subject with a chewing gum composition disclosed herein. In an aspect, a method is provided for protecting dentin from acid-mediated degradation of a subject, the method includes providing a chewing gum composition disclosed herein. In an embodiment, the subject is a mammal (e.g., human). In some embodiments, the subject is an orthodontic patient, a child, or a subject with cognitive motor impairments. In an embodiments, the subject is a non-human animal such as a companion animal and/or livestock.

In another aspect, a method is provided for maintaining or increasing the systemic health of a subject (such as a mammal), the method including providing a composition to an oral surface of the subject at least once a day for a duration of time, wherein the composition comprises the chewing gum composition as disclosed herein.

The present disclosure is illustrated and further described in more detail with reference to the following non-limiting examples.

### EXAMPLES

### Example 1

Cq averages were obtained for a variety of different bacteria in contact with compositions of embodiments of the present disclosure. Cq averages (a Cq value, or cycle threshold value, is the number of times a machine copies a piece of genetic material before it can be detected in a PCR test) for S. mutans (pathogenic), S. anginosus (pathogenic), S. salivarius (commensal in newborns), and S. sanguinis (commensal in adults) under each prebiotic condition. Statistical significance: In the S. mutans group, the Cq values of hydroxyapatite, sodium bicarbonate, and combined L-arginine with sodium bicarbonate. In the S. anginosus group, the Cq values of L-arginine, sodium bicarbonate, and the combined L-arginine with sodium bicarbonate. In the S. salivarius group, the Cq values of hydroxyapatite, L-arginine, sodium bicarbonate, and the combined L-arginine with sodium bicarbonate. In the S. sanguinis group, the Cq values of Xylitol, hydroxyapatite, and the combined coconut oil with xylitol.

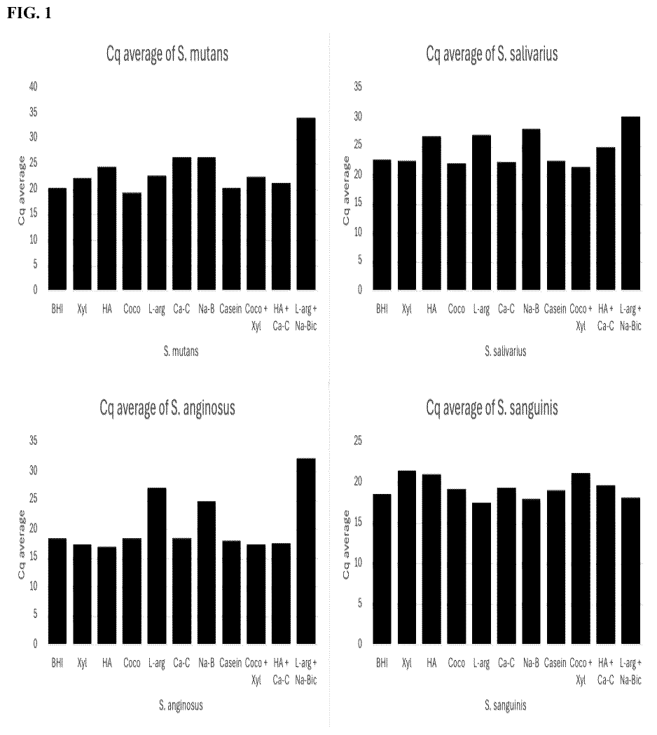

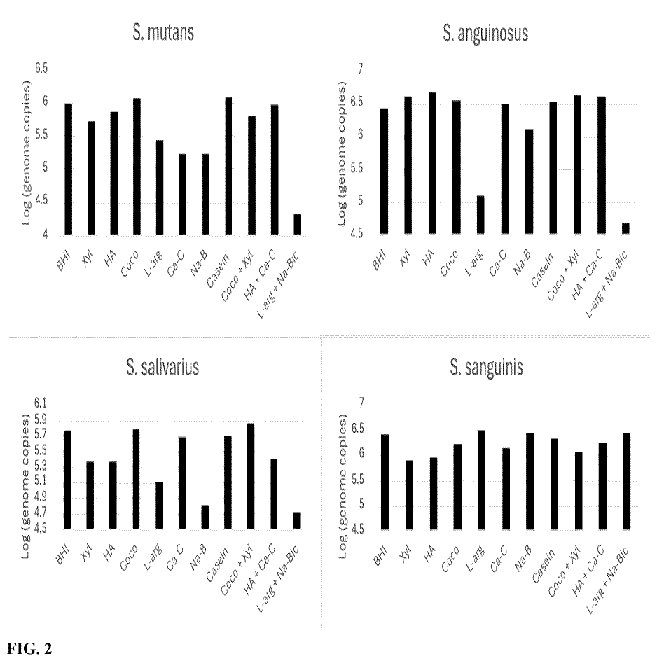

Results are shown in FIGS. 1 and 2. FIG. 1 shows Cq averages of S. mutans (pathogenic), S. anginosus (pathogenic), S. salivarius (commensal in newborns), and S. sanguinis (commensal in adults) after contact with the described compositions. FIG. 2 shows genome copy data of S. mutans (pathogenic), S. anginosus (pathogenic), S. salivarius (commensal in newborns), and S. sanguinis (commensal in adults) after contact with the described compositions.

L-arginine emerged as a particularly beneficial prebiotic, as it was able to increase the growth of S. sanguinis, a commensal bacterium associated with oral health, while simultaneously reducing the growth of pathogenic species like S. anginosus to a significant extent.

Sodium bicarbonate showed a significant effect in reducing the levels of S. mutans and S. anginosus, particularly when combined with L-arginine. The synergistic effect of L-arginine and sodium bicarbonate resulted in an even more pronounced reduction of pathogenic bacteria compared to either prebiotic alone. Certain commensal oral bacteria metabolize L-arginine via the arginine deiminase system, producing ammonia, which neutralizes acids generated by acidogenic species. This alkalinizing effect shifts the biofilm environment away from conditions favorable to S. mutans and other acid-tolerant organisms. Sodium bicarbonate also demonstrated significant inhibitory effects on cariogenic bacteria, consistent with its established buffering capacity. By reacting with hydrogen ions, sodium bicarbonate rapidly elevates biofilm pH and counteracts post-sugar acidification, which is particularly relevant for subjects that experience increased plaque accumulation. Importantly, the combination did not reduce S. sanguinis levels, which supports that this prebiotic combination could help limit harmful bacteria without negatively affecting commensal species.

The combination of coconut oil and xylitol promoted the growth of S. sanguinis without significantly affecting cariogenic bacteria. This suggests that coconut oil and xylitol may selectively support commensal bacteria while not exacerbating the growth of harmful species.

The genome copies data were not used for statistical analysis, purely for visual appreciation.

L-arginine combined with sodium bicarbonate has a positive effect on decreasing both pathogenic bacteria (S. mutans and S. anguinosus) while having no effect on commensal bacteria (S. sanguinis).

### Example 2

A chewing gum formulation is prepared comprising (by weight of the final gum): about 0.5% to about 10% L-arginine, about 0.5% to about 35% sodium bicarbonate, and about 0.5% to about 20% hydroxyapatite, with a sugar-free gum base and sweeteners. Hydroxyapatite is provided as nano-hydroxyapatite (e.g., 50-200 nm average particle size). In some runs, sodium bicarbonate is provided as a coated particle to reduce premature interaction with moisture during processing.

The gum is evaluated in an in vitro plaque biofilm model. After a carbohydrate challenge, plaque pH is monitored over time with and without gum chewing. The gum is observed to increase plaque pH relative to control gum and to shorten the time plaque pH remains below the critical demineralization threshold (e.g., pH 5.5).

Enamel or hydroxyapatite disk specimens are exposed to the biofilm model with repeated acid challenges. Specimens treated with the gum exhibit increased surface microhardness and/or reduced lesion depth relative to controls, consistent with remineralization support.

### Example 3

An edible animal (e.g., dog) dental chew is prepared comprising (by weight of the chew): about 0.1% to about 5% L-arginine (or an arginine salt), about 0.5% to about 10% sodium bicarbonate, and about 0.1% to about 10% hydroxyapatite in a starch/protein matrix. The chew is formulated without xylitol.

Dogs are administered one or more chews daily for 4 to 8 weeks. Oral health endpoints include plaque index, gingival index, halitosis scoring, and/or microbial profiling from oral swabs. Dogs receiving the chew exhibit reduced plaque accumulation and improved breath score compared to control chews.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

All statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Various other components may be included and called upon for providing for aspects of the teachings herein. For example, additional materials, combinations of materials and/or omission of materials may be used to provide for added embodiments that are within the scope of the teachings herein. Adequacy of any particular element for practice of the teachings herein is to be judged from the perspective of a designer, manufacturer, seller, user, system operator or other similarly interested party, and such limitations are to be perceived according to the standards of the interested party.

In the disclosure hereof any element expressed as a means for performing a specified function is intended to encompass any way of performing that function including, for example, a) a combination of circuit elements and associated hardware which perform that function or b) software in any form, including, therefore, firmware, microcode or the like as set forth herein, combined with appropriate circuitry for executing that software to perform the function. Applicants thus regard any means which can provide those functionalities as equivalent to those shown herein. No functional language used in claims appended herein is to be construed as invoking 35 U.S.C. § 112(f) interpretations as “means-plus-function” language unless specifically expressed as such by use of the words “means for” or “steps for” within the respective claim.

When introducing elements of the present disclosure or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements. The term “exemplary” is not intended to be construed as a superlative example but merely one of many possible examples.

While the disclosure has been described with reference to an exemplary embodiment, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiment disclosed as the best mode contemplated for carrying out this disclosure, but that the disclosure will include all embodiments falling within the scope of the appended claims. Any combination of the above-described elements in all possible variations thereof is encompassed by the disclosure unless otherwise indicated herein or otherwise clearly contradicted by context.

For reasons of completeness, various aspects of the disclosure are set out in the following numbered clauses:
Clause 1. A composition comprising a basic amino acid (e.g., L-arginine), an alkali-generating or buffering system (e.g., a bicarbonate salt such as sodium bicarbonate), and hydroxyapatite.
Clause 1A: A composition comprising L-arginine, a bicarbonate salt, and hydroxyapatite.
Clause 2. The composition of clause 1, wherein the bicarbonate salt is selected from sodium bicarbonate, potassium bicarbonate, calcium bicarbonate, magnesium bicarbonate, and ammonium bicarbonate.
Clause 3. The composition of clause 1 or 2, wherein the composition is chewing gum.
Clause 4. The composition of any one of clauses 1 to 3, wherein the composition is an oral care product.
Clause 5. The composition of any one of the preceding clauses, wherein the bicarbonate salt is sodium bicarbonate and wherein the composition comprises from about 0.01% to about 15% by weight of L-arginine, from about 0.1% to about 50% by weight of sodium bicarbonate, and from about 0.01% to about 30% by weight of hydroxyapatite.
Clause 6. The composition of clause 5, wherein the composition comprises from about 0.5% to 10% by weight of L-arginine, from about 0.5% to about 35% by weight of sodium bicarbonate, and from about 0.5% to about 20% by weight of hydroxyapatite.
Clause 7. The composition of clause 6, wherein the composition comprises from about 0.01% to about 5% by weight of L-arginine, from about 0.1% to about 25% by weight of sodium bicarbonate, and from about 0.01% to about 15% by weight of hydroxyapatite.
Clause 8. The composition of any one of the preceding clauses, wherein the L-arginine and sodium bicarbonate are present in the composition at a weight ratio of about 1:4.
Clause 9. The composition of any one of the preceding claims, wherein the composition comprises from about 10% to about 95% by weight gum base.
Clause 10. The composition of clause 9, wherein the gum base comprises one or more components selected from elastomers, solvents, plasticizers, fillers, flavoring agents, coloring agents, and sweetening agents.
Clause 11. The composition of clause 10, wherein the elastomers are selected from chicle, jelutong, gutta percha, guayale, crown gum, butadiene-styrene copolymers, isobutylene-isoprene copolymers, polyethylene, polyisobutylene, polyvinyl acetate, or any combinations thereof.
Clause 12. The composition of clause 10 or 11, wherein the elastomers are present in the composition at a concentration of from about 14% to about 50% by weight.
Clause 13. The composition of clause 12, wherein the elastomers are present in the composition at a concentration of from about 20% to about 30% by weight.
Clause 14. The composition of any one of the preceding clauses, wherein the composition further comprises one or more elastomer solvents.
Clause 15. The composition of clause 14, wherein the elastomer solvents are selected from methyl, glycerol or pentaerythritol esters of rosins or modified rosins.
Clause 16. The composition of clause 15, wherein the modified rosins are hydrogenated, dimerized, polymerized rosins, or any combination thereof.
Clause 17. The composition of any one of the preceding clauses, wherein the composition further comprises a terpene resin.
Clause 18. The composition of any one of clauses 14 to 17, wherein the elastomer solvent is present at a concentration of about 10% to about 75% by weight.
Clause 19. The composition of clause 18, wherein the elastomer solvent is present in the composition at a concentration of about 15% to about 50% by weight.
Clause 20. The composition of any one of the preceding clauses, wherein the composition further comprises one or more additional components selected from lanolin, lecithin, glycerol monostearate, stearic acid, glycerol triacetate, triacetin, glycerin, natural waxes, petroleum waxes, microcrystalline waxes, lard, tallow, soybean oil, cottonseed oil, hydrogenated vegetable oil, partially hydrogenated vegetable oil and cocoa butter, wherein the additional components are present in the composition and a concentration of up to about 30% by weight.
Clause 21. The composition of any one of the preceding clauses, wherein the composition comprises from about 10% to about 95% by weight of confectionery base.
Clause 22. The composition of any one of the preceding clauses, wherein the composition comprises a sweetener.
Clause 23. The composition of clause 22, wherein the sweetener is selected from sucrose, dextrose, maltose, fructose, aspartame, acesulfame salts, alitame saccharin, sorbitol, mannitol, xylitol, maltitol, isomalt, erythritol, hydrogenated starch hydrolysates, and coconut oil.
Clause 24. The composition of any one of the preceding clauses, wherein the composition further comprises a coloring agent.
Clause 25. The composition of any one of the preceding clauses, wherein the composition further comprises a filler selected from dicalcium phosphate, magnesium carbonate, aluminum hydroxide, alumina, aluminum silicates, talc, calcium carbonate, cellulose, and combinations thereof, wherein the filler is present in composition at a concentration of from about 5% to about 35% by weight.
Clause 26. The composition of any one of the preceding clauses, wherein the composition further comprises one or more of a chemical whitening agent, an opaque whitening agent, an anticalculus agent, a surfactant system that comprises sodium lauryl sulfate and an N-acyl taurate, a stannous ion agent; a fluoride compound, sodium fluoride, chlorhexidine, alexidine; hexetidine, sanguinarine, benzalkonium chloride, salicylanilide, domiphen bromide, cetylpyridinium chloride (CPC), tetradecyl pyridinium chloride (TPC), N-tetradecyl-4-ethylpyridinium chloride (TDEPC), octenidine, delmopinol, octapinol, nisin, zinc ion, copper ion, essential oils, furanones, bacteriocins, ethyllauroyl arginate, extracts of magnolia, a metal ion source, arginine bicarbonate, honokiol, magnolol, ursolic acid, tirsic acid, morin, extract of sea buckthorn, a peroxide, an enzyme, a camellia extract, a flavonoid, a flavin, halogenated diphenyl ether, creatine, propolis, or any combinations thereof.
Clause 27. A kit or system comprising the composition of any one of clauses 1 to 26.
Clause 28. A method for improving oral health or hygiene, comprising:
   providing the composition of any one of clauses 1 to 26 or the kit or system of clause 27to a subject in need thereof.
Clause 29. The method of clause 28, wherein the composition is contacted with an oral surface of the subject.
Clause 30. The method of clause 28 or 29, wherein the providing reduces dental sensitivity in the subject, reduces the incidence of dental caries in the subject, reduces dental plaque in the subject, whitens teeth in the subject, prevents tooth demineralization in the subject, promotes tooth remineralization in the subject, improves overall health in the subject, or any combination thereof.
Clause 31. The method of any one of clauses 28 to 30, wherein the subject is a human.
Clause 32. The method of any one of clauses 28 to 31, wherein the subject is an orthodontic patient, a child, or a subject with cognitive motor impairments.
Clause 33. The use of the composition of any one of clauses 1 to 26 or the kit or system of clause 27 to improve oral health or hygiene.
Clause 34. The method of any one of clauses 28 to 32, wherein the subject is a companion animal or livestock.
Clause 35. The composition of clause 1, wherein the L-arginine is present as a salt selected from arginine bicarbonate, arginine carbonate, arginine citrate, arginine acetate, arginine lactate, arginine phosphate, arginine hydrochloride, and combinations thereof.
Clause 36. The composition of clause 1, wherein the bicarbonate salt further comprises a carbonate salt and/or a phosphate salt.
Clause 37. The composition of clause 1, wherein the hydroxyapatite is nano-hydroxyapatite having an average particle size of about 20 nm to about 900 nm.
Clause 38. The composition of clause 1, wherein the hydroxyapatite comprises fluorohydroxyapatite and/or fluorapatite.
Clause 39. The composition of clause 1, wherein one or more of (i) the L-arginine, (ii) the bicarbonate salt, and (iii) the hydroxyapatite is encapsulated or coated to provide staged release in the oral cavity.
Clause 40. The composition of clause 1, wherein the chewing gum comprises a coating and wherein at least a portion of one or more of the L-arginine, bicarbonate salt, and hydroxyapatite is present in the coating.
Clause 41. The composition of clause 1, further comprising one or more calcium and/or phosphate ion donors selected from calcium glycerophosphate, dicalcium phosphate, calcium lactate, calcium citrate, sodium phosphate, and combinations thereof.
Clause 42. The composition of clause 1, wherein the composition is formulated as a lozenge, mint, chewable tablet, oral dissolvable film, or oral powder.
Clause 43. The method of clause 28, wherein providing the composition comprises chewing or dissolving the composition for at least about 5 minutes.
Clause 44. The method of clause 28, wherein providing the composition is performed after a meal or snack to reduce post-prandial plaque acidification.
Clause 45. The method of clause 28, wherein providing the composition raises plaque pH above about 5.5 within about 10 minutes of administration.
Clause 46. The method of clause 28, wherein providing the composition raises oral pH above about 5.5 within about 10 minutes of administration.
Clause 47. The method of clause 28, wherein the subject is a companion animal or livestock and the composition is provided as an edible chew or treat.
Clause 48. A method of making a chewing gum composition of claim 1, comprising mixing at least a portion of hydroxyapatite with chewing gum additives to form a premix and combining the premix with a gum base, wherein the bicarbonate salt is added in dry form.
Clause 49. Use of the composition of clause 1 for promoting a balanced oral microbiome by reducing acidogenic and/or aciduric bacteria while maintaining one or more commensal oral bacteria.

### Incorporation by Reference

All U.S. and PCT patent publications and U.S. patents mentioned herein are hereby incorporated by reference in their entirety as if each individual patent publication or patent was specifically and individually indicated to be incorporated by reference. In case of conflict, the present application, including any definitions herein, will control.

### Other Embodiments

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A composition comprising L-arginine, a bicarbonate salt, and hydroxyapatite.

2. The composition of claim 1, wherein the composition is chewing gum.

3. The composition of claim 1, wherein the composition is an oral care product.

4. The composition of claim 1, wherein the bicarbonate salt is sodium bicarbonate.

5. The composition of claim 4, wherein the composition comprises from about 0.01% to 15% by weight of L-arginine, from about 0.1% to about 50% by weight of sodium bicarbonate, and from about 0.01% to about 30% by weight of hydroxyapatite.

6. The composition of claim 5, wherein the composition comprises from about 0.5% to 10% by weight of L-arginine, from about 0.5% to about 35% by weight of sodium bicarbonate, and from about 0.5% to about 20% by weight of hydroxyapatite.

7. The composition of claim 6, wherein the composition comprises from about 0.01% to about 5% by weight of L-arginine, from about 0.1% to about 25% by weight of sodium bicarbonate, and from about 0.01% to about 15% by weight of hydroxyapatite.

8. The composition of claim 4, wherein the L-arginine and sodium bicarbonate are present in the composition at a weight ratio of about 1:4.

9. The composition of claim 1, wherein the composition comprises from about 10% to about 95% by weight gum base.

10. The composition of claim 9, wherein the gum base comprises one or more components selected from elastomers, solvents, plasticizers, fillers, flavoring agents, coloring agents, and sweetening agents.

11. The composition of claim 1, wherein the composition further comprises one or more additional components selected from lanolin, lecithin, glycerol monostearate, stearic acid, glycerol triacetate, triacetin, glycerin, natural waxes, petroleum waxes, microcrystalline waxes, lard, tallow, soybean oil, cottonseed oil, hydrogenated vegetable oil, partially hydrogenated vegetable oil and cocoa butter, wherein the additional components are present in the composition and a concentration of up to about 30% by weight.

12. The composition of claim 1, wherein the composition comprises from about 10% to about 95% by weight of confectionery base.

13. The composition of claim 1, wherein the composition further comprises one or more of a chemical whitening agent, an opaque whitening agent, an anticalculus agent, a surfactant system that comprises sodium lauryl sulfate and an N-acyl taurate, a stannous ion agent; a fluoride compound, sodium fluoride, chlorhexidine, alexidine; hexetidine, sanguinarine, benzalkonium chloride, salicylanilide, domiphen bromide, cetylpyridinium chloride (CPC), tetradecyl pyridinium chloride (TPC), N-tetradecyl-4-ethylpyridinium chloride (TDEPC), octenidine, delmopinol, octapinol, nisin, zinc ion, copper ion, essential oils, furanones, bacteriocins, ethyllauroyl arginate, extracts of magnolia, a metal ion source, arginine bicarbonate, honokiol, magnolol, ursolic acid, tirsic acid, morin, extract of sea buckthorn, a peroxide, an enzyme, a camellia extract, a flavonoid, a flavin, halogenated diphenyl ether, creatine, propolis, or any combinations thereof.

14. A kit or system comprising the composition of claim 1.

15. A method for improving oral health or hygiene, comprising:
providing the composition of claim 1 to a subject in need thereof.

16. The method of claim 15, wherein the composition is contacted with an oral surface of the subject.

17. The method of claim 15, wherein the providing reduces dental sensitivity in the subject, reduces the incidence of dental caries in the subject, reduces dental plaque in the subject, whitens teeth in the subject, prevents tooth demineralization in the subject, promotes tooth remineralization in the subject, improves overall health in the subject, or any combination thereof.

18. The method of claim 15, wherein the subject is a human.

19. The method of claim 15, wherein the subject is an orthodontic patient, a child, or a subject with cognitive motor impairments.

20. The method of claim 15, wherein the subject is a companion animal or livestock.
